(19) United States
(12) Patent Application Publication
(10) Pub. No.: US 2005/0112686 A1
(43) Pub. Date: May 26, 2005

(54) Method for introducing molecules into biological samples

(76) Inventors: John H. Crowe, Davis, CA (US); Fern Tablin, Davis, CA (US); Willem Wolkers, Davis, CA (US); Ann E. Oliver, Sacramento, CA (US); Kamran Jamil, Woodland Hills, CA (US); Joong-Hyuck Auh, Davis, CA (US); Thurein Htoo, Rochester, NY (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/722,154

(22) Filed: Nov. 25, 2003

(51) Int. Cl. G01N 33/53; C12Q 1/34

(52) U.S. Cl. 435/7.1; 435/317.1; 435/18

(57) ABSTRACT

A method for loading a biological sample comprising loading a biological sample with a solute by fluid phase endocytosis to produce an internally loaded biological sample. Within the biological sample a first matter (e.g., a vesicle) having the solute fuses with a second matter (e.g., a lysosome) to produce a fused matter containing the solute. Loading of the biological sample includes transferring the solute from the fused matter into cytoplasm within the biological sample.

### FIELD OF THE INVENTION

Embodiments of the present invention generally broadly relate to biological samples, such as mammalian cells, platelets, and the like. More specifically, embodiments of the present invention generally provide for the preservation and survival of biological samples.

Embodiments of the present invention also generally broadly relate to the therapeutic uses of biological samples; more particularly to manipulations or modifications of biological samples, such as loading biological samples with solutes (e.g., carbohydrates, such as trehalose) and preparing dried compositions that can be re-hydrated at the time of application. When biological samples for various embodiments of the present invention are re-hydrated, they are immediately restored to viability.

The compositions and methods for embodiments of the present invention are useful in many applications, such as in medicine, pharmaceuticals, biotechnology, and agriculture, and including transfusion therapy, as hemostasis aids and for drug delivery.

Statement Regarding Federal Sponsored Research and Development

Embodiments of this invention were made with Government support under Grant No. N66001-03-1-8927, awarded by the Department of Defense Advanced Research Projects Agency (DARPA). Further embodiments of this invention were made with Government support under Grant Nos. HL57810 and HL61204, awarded by the National Institutes of Health. The Government has certain rights to embodiments of this invention.

### BACKGROUND OF THE INVENTION

A biological sample includes cells and blood platelets. A cell is typically broadly regarded in the art as a small, typically microscopic, mass of protoplasm bounded externally by a semi-permeable membrane, usually including one or more nuclei and various other organelles with their products. A cell is capable either alone or interacting with other cells of performing all the fundamental function(s) of life, and forming the smallest structural unit of living matter capable of functioning independently.

Cells may be transported and transplanted; however, this requires preservation which includes drying (e.g., vacuum drying, air drying, etc.), freezing and subsequent reconstitution (e.g., thawing, re-hydration, etc.) after transportation. Unfortunately, a very low percentage of cells retain their functionality after undergoing freezing and thawing. While some protectants, such as the cryoprotectant such as dimethylsulfoxide, tend to lessen the damage to cells, they still do not prevent some loss of cell functionality.

Blood platelets are typically generally oval to spherical in shape and have a diameter of 2-4 μm. Today platelet rich plasma concentrates are stored in blood bags at 22° C.; however, the shelf life under these conditions is limited to five days. The rapid loss of platelet function during storage and risk of bacterial contamination complicates distribution and availability of platelet concentrates. Platelets tend to become activated at low temperatures. When activated they are substantially useless for an application such as transfusion therapy. Therefore, the development of preservation methods that will increase platelet lifespan is desirable.

Trehalose has been found to be suitable in the preservation of cells and platelets. Trehalose is a disaccharide found at high concentrations in a wide variety of organisms that are capable of surviving almost complete dehydration. Trehalose has been shown to stabilize membranes, proteins, and certain cells and platelets during drying (e.g., freeze-drying) in vitro.

Spargo et al., U.S. Pat. No. 5,736,313, issued Apr. 7, 1998, have described a method in which platelets are loaded overnight with an agent, preferably glucose, and subsequently lyophilized. The platelets are preincubated in a buffer and then are loaded with carbohydrate, preferably glucose, having a concentration in the range of about 100 mM to about 1.5 M. The incubation is taught to be conducted at about 10° C. to about 37° C., most preferably about 25° C.

U.S. Pat. No. 5,827,741, Beattie et al., issued Oct. 27, 1998, discloses cryoprotectants for human cells and platelets, such as dimethylsulfoxide and trehalose. The cells or platelets may be suspended, for example, in a solution containing a cryoprotectant at a temperature of about 22° C. and then cooled to below 15° C. This incorporates some cryoprotectant into the cells or platelets, but not enough to prevent hemolysis of a large percentage of the cells or platlets.

Accordingly, a need exists for the effective and efficient preservation of biological samples, such as platelets and cells, and the like. More specifically, and accordingly further, a need also exists for the effective and efficient preservation of platelets and cells (e.g., erythrocytic cells, eukaryotic cells, or any other cells, and the like), such that the preserved platelets and cells respectively maintain their biological properties and may readily become viable after storage.

### SUMMARY OF EMBODIMENT OF THE INVENTION

Embodiments of the present invention provide a process for loading a biological sample comprising loading a biological sample with a solute (e.g., trehalose) by fluid phase endocytosis to produce an internally loaded biological sample. The loading of a biological sample by fluid phase endocytosis comprises fusing within the biological sample a first matter (e.g., a vesicle) with a second matter (a lysosome) to produce a fused matter. The fused matter preferably comprises the solute. The loading of a biological sample by fluid phase endocytosis additionally comprises transferring the solute from the fused matter into a cytoplasm within the biological sample. The fused matter may comprise a lower pH than a pH of the first matter. The fused matter preferably comprises a pH of less than about 6.5, such as from about 3.0 to about 6.0. The biological sample may include a biological sample selected from a group of biological samples comprising a platelet and a cell.

Embodiment of the present invention also provide a process for loading a solute into a biological sample comprising forming within a biological sample a vesicle having a solute, and lowering the pH of the vesicle to cause the biological sample to be loaded with the solute. The lowering of the pH of the vesicle comprises fusing the vesicle with a lysosome to produce fused matter. The lowering of the pH of the vesicle may also comprise increasing the permeability of a membrane in the biological sample for facilitating the passage of the solute from the vesicle into the biological sample. The fused matter preferably comprises a pH of less that about 6.5, such as from about 3.0 to about 6.0. A biological sample produced in accordance with the foregoing process is also provided by embodiments of the present invention.

Embodiments of the present invention also further provide a process for preparing a dehydraded biological sample comprising providing a biological sample selected from a mammalian species, loading the biological sample with a solute by fluid phase endocytosis to produce a loaded biological sample, and drying (e.g., vacuum drying, air drying, freeze-drying, etc.) the loaded biological sample to produce a dehydrated biological sample.

These provisions together with the various ancillary provisions and features which will become apparent to those skilled in the art as the following description proceeds, are attained by the processes and biological samples (e.g., platelets, eukaryotic cells, and erythrocytic cells) of the present invention, preferred embodiments thereof being shown with reference to the accompanying drawings, by way of example only, wherein:

### BRIEF DESCRIPTION OF THE DRAWINGS

FIG. 1 is an exemplary diagram of a biological sample having a plasma membrane with an internal protein coating and encapsulating a cytoplasm having lysosomes and a nucleus;

FIG. 2 is an elevational view of the plasma membrane in contact with a solute solution having a solute which is to be loaded into the biological sample;

FIG. 3 is an elevational view of the plasma membrane in the process of being loaded with a solute;

FIG. 4 is an elevational view of a vesicle containing a solute and connected to the plasma membrane;

FIG. 5 is a diagram of the cytoplasm having a lysosome and a vesicle containing a solute and which “budded off” or released from the plasma membrane;

FIG. 6 is a diagram of a lysosome fused with a vesicle to produce fused matter or material containing a solute;

FIG. 7 is a diagram of the fused matter or material containing a solute which is in the process of passing in direction of the arrow from the fused matter or material into the cytoplasm of the biological sample to effectively load the biological sample with the solute;

FIG. 8 is an enlarged chemical structural, chain formula diagram of trehalose, a non-reducing disaccharide of glucose, with an arrow pointing to a glycosidic bond;

FIG. 9 is an enlarged chemical structural, chain formula diagram of sucrose, a non-reducing disaccharide of glucose and fructose, with an arrow pointing to a glycosidic bond which is much more susceptible to hydrolysis than the glycosidic bond in trehalose;

FIG. 10 is a graph of pH vs. % intact (i.e., % non-degraded) for trehalose and sucrose;

FIG. 11 is a graph of % leakage of a fluorescent dye, carboxyfluorescein (CF), from phospholipid vesicles as a function of pH and time;

FIG. 12 is a graph of rates of leakage (% leakage/10 minutes) as a function of pH;

FIG. 13 is a graph of projected time to achieve 100% leakage, based on FIGS. 20 and 21, as a function of pH;

FIG. 14 is a picture of control cells at zero (0) incubations time, showing no leakage of Lucifer yellow dye into the cytoplasm of the control cell;

FIG. 15 is a picture of cells after 1 hour incubation time, showing Lucifer yellow dye in punctate structures (i.e., endocytotic vesicles) with some leakage of Lucifer yellow dye into the cytoplasm;

FIG. 16 is a picture of cells after 3.5 hours incubation time, showing Lucifer yellow dye in punctuated structures (i.e., endocytotic vesicles) with more leakage of Lucifer yellow dye into the cytoplasm than the leakage represented in the picture of FIG. 24; and

FIG. 17 is a picture of cells after 5.0 hours incubation time, showing a uniform stain of Lucifer yellow dye which suggests that Lucifer yellow dye has leaked into the cytoplasm.

### DETAILED DESCRIPTION OF PREFERRED EMBODIMENTS OF THE INVENTION

Embodiments of the present invention broadly include biological samples, preferably mammalian biological samples. Embodiments of the present invention further broadly include methods for preserving biological samples, as well as biological samples that have been manipulated (e.g., by drying to produce dehydrated biological samples) or modified (e.g., loaded with a chemical or drug) in accordance with methods of the present invention. Embodiments of the present invention also further broadly include methods for increasing the survival of biological samples, especially during drying and following drying, storing and rehydrating.

Biological samples for various embodiments of the present invention comprise any suitable biological sample, such as blood platelets and cells. The cells may be any type of cell including, not by way of limitation, erythrocytic cells, eukaryotic cells or any other cell, whether nucleated or non-nucleated.

The term “erythrocytic cell” is used to mean any red blood cell. Mammalian, particularly human, erythrocytes are preferred. Suitable mammalian species for providing erythrocytic cells include by way of example only, not only human, but also equine, canine, feline, or endangered species.

The term “eukaryotic cell” is used to mean any nucleated cell, i.e., a cell that possesses a nucleus surrounded by a nuclear membrane, as well as any cell that is derived by terminal differentiation from a nucleated cell, even though the derived cell is not nucleated. Examples of the latter are terminally differentiated human red blood cells. Mammalian, and particularly human, eukaryotes are preferred. Suitable mammalian species include by way of example only, not only human, but also equine, canine, feline, or endangered species.

The source of the eukaryotic cells may be any suitable source such that the eukaryotic cells may be cultivated in accordance with well known procedures, such as incubating the eukaryotic cells with a suitable serum (e.g., fetal bovine serum). After the eukaryotic cells are cultured, they are subsequently harvested by any conventional procedure, such as by trypsinization, in order to be loaded with a protective preservative. The eukaryotic cells are preferably loaded by growing the eukaryotic cells in a liquid tissue culture medium. The preservative (e.g., an oligosaccharide, such as trehalose) is preferably dissolved in the liquid tissue culture medium, which includes any liquid solution capable of preserving living cells and tissue. Many types of mammalian tissue culture media are known in the literature and available from commercial suppliers, such as Sigma Chemical Company, St. Louis, Mo., USA: Aldrich Chemical Company, Inc., Milwaukee, Wis., USA; and Gibco BRL Life Technologies, Inc., Grand Island, N.Y., USA. Examples of media that are commercially available are Basal Medium Eagle, CRCM-30 Medium, CMRL Medium-1066, Dulbecco's Modified Eagle's Medium, Fischer's Medium, Glasgow Minimum Essential Medium, Ham's F-10 Medium, Ham's F-12 Medium, High Cell Density Medium, Iscove's Modified Dulbecco's Medium, Leibovitz's L-15 Medium, McCoy's 5A Medium (modified), Medium 199, Minimum Essential Medium Eagle, Alpha Minimum Essential Medium, Earle's Minimum Essential Medium, Medium NCTC 109, Medium NCTC 135, RPMMI-1640 Medium, William's Medium E, Waymouth's MB 752/1 Medium, and Waymouth's MB 705/1 Medium.

Broadly, the preparation of solute-loaded biological sample(s) (e.g., platelets and cells) in accordance with embodiments of the invention comprises the steps of loading one or more biological samples with a solute by placing the biological samples in a solute solution for transferring by fluid phase endocytosis the solute from the solution into the biological sample(s). For increasing the transfer or uptake of the solute from the solute solution, the solute solution temperature, or incubation temperature, may have a temperature above about 25° C., more preferably above 30° C., such as from about 30° C. to about 40° C.

The solute solution may be any suitable physiologically acceptable solution in an amount and under conditions effective to cause uptake or “introduction” of the solute from the solute solution into the biological sample(s) for fluid phase endocytosis. A physiologically acceptable solution is a suitable solute-loading buffer, such as any of the buffers stated in the previously mentioned related patent applications, all having been incorporated herein by reference thereto.

The solute is preferably a carbohydrate (e.g., an oligosaacharide) selected from the following groups of carbohydrates: a monosaccharide, an oligosaccharide (e.g., bioses, trioses, tetroses, pentoses, hexoses, heptoses, etc), a disaccharide (e.g., lactose, maltose, sucrose, melibiose, trehalose, etc), a trisaccharide (e.g., raffinose, melezitose, etc), or tetrasaccharides (e.g., lupeose, stachyose, etc), and a polysaccharide (e.g., dextrins, starch groups, cellulose groups, etc). More preferably, the carbohydrate is a disaccharide, with trehalose being the preferred, particularly since it has been discovered that trehalose does not degrade or reduce in complexity upon being loaded. Thus, in the practice of various embodiments of the invention, trehalose is transferred from a solution into the biological sample without degradation of the trehalose.

Loading of the solute from the solute solution into the biological sample(s) broadly includes producing and/or forming at least a portion of a biological membrane of the microbiological sample(s) to entrap and include a solute; and fusing, commingling, or otherwise combining in any suitable manner, the produced and/or formed solute-containing portion of the biological membrane with a lysosome to produce fused-matter from which the solute is transferred into the cytoplasm of the biological membrane (e.g., a cell). Producing and/or forming at least a portion of the biological membrane to include the solute comprises transferring or passing the solute from the solute solution against and/or into a portion of the biological membrane for producing and/or forming a vesicle (i.e., an endosomal, phagocytic vesicle) containing the solute. The vesicle after a period of time, which depends on the residence time of the biological sample in the solute solution, subsequently breaks or severs (i.e., “buds off”) from the biological membrane into the cytoplasm of the biological sample(s) to fuse with lysosome(s).

The fusing or combining of the vesicle with a lysosome is caused by recognition sites on both membranes that promote fusion or the combining. The produced fused matter subsequently breaks down or degrades, with the lysosomal membranes being recycled and reloaded in the Golgi. Most sugars are degraded in the lysosome to monosaccharides, which are then transferred to the cytoplasm for further degradation. It is suggested that the mechanism of transfer includes the magnitude of the internal pH in the lysosomes which leads to leakage across the bilayers. The lysosome(s) has/have a low pH, such as a pH ranging from about from about 3.0 to about 5.0. In addition there is the presence of acidic hydrolases in the lysosomes. The vesicle, especially when the vesicle contains the solute, has a higher pH than the pH of the lysosome(s). The vesicle typically has a pH ranging from about 7.0 to about 8.0. Thus, the internal, engulfed material within the fused matter contains a reduced pH, a pH lower than the pH of the vesicle (e.g., a pH less than about 6.5, such as a pH ranging from about 3.5 to about 6.0).

The reduced pH, an acidic pH, causes the membrane of the produced fused matter to have an increased permeability. Stated alternatively, lowering the pH of the internal, engulfed material through the fusing of lysosome and vesicles produces an acidic engulfed material within the fused matter, which concomitantly raises or increases the permeability of the membrane of the fused matter. With an increase in permeability, the solute (or any low molecular weight molecules) leaks or passes through the membrane of the fused matter and into the cytoplasm.

When the solute is a sugar, most sugars hydrolyze within the fused matter. An exception is trehalose, which escapes degradation due to the stability of its associated glycosidic linkage. The broken down components of the lysosome and the vesicles are released into the cytoplasm for further metabolism. The components of sucrose would include glycose and fructose, which are degraded by the well known glycolytic pathway and the TCA cycle to CO₂ and H₂O. Because trehalose remains in tact for effecting the transferring and the loading of the solute into the cytoplasm of the biological sample(s), and does not degrade in conditions found in the lysome-endosome, trehalose is a preferred solute. However, it is to be understood that while trehalose is a preferred solute, the spirit and scope of the present invention includes any solute comprising one or more molecules that survive the environmental conditions within the fused matter. More specifically, the solute for various embodiments of the present invention comprises one or more of any molecule(s) that does not degrade under the transferring or loading conditions, or within the environmental conditions within the fused matter resulting from the fusing of lysosome and the vesicle. After the solute is transferred out of the fused matter and into the cytoplasm, stability is conferred on the biological sample for further treatment or processing, such as drying.

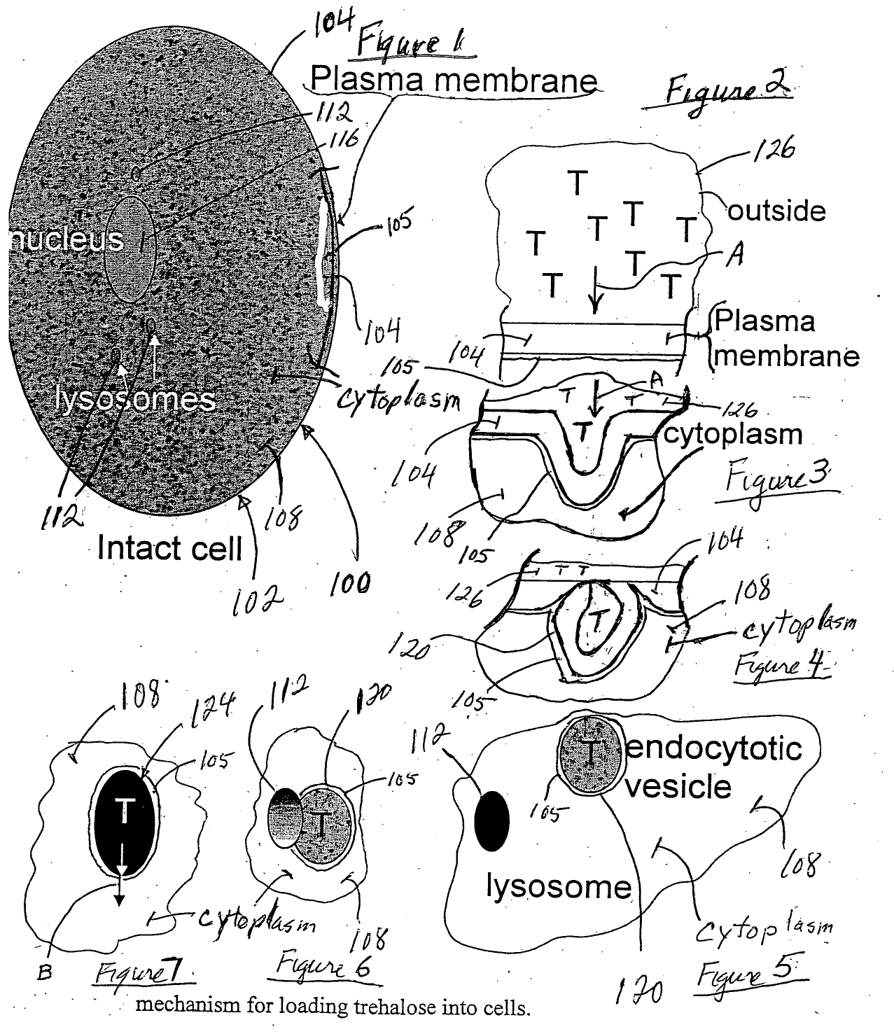

Referring now to FIGS. 1-7 for more specifically describing an embodiment of a mechanism for loading by fluid phase endocytosis a solute from a solute solution into a biological sample (e.g., platelet(s), cell(s), etc.), there is seen in FIG. 1 a biological sample 100 which is exemplarily represented as an intact cell 102 having a plasma membrane 104 internally coated with a protein (e.g., clathrin) 105. The plasma membrane 104 encapsulates cytoplasm 108 having lysosomes 112. The plasma membrane 104 may also encapsulate a nucleus 116 contained within the cytoplasm 108.

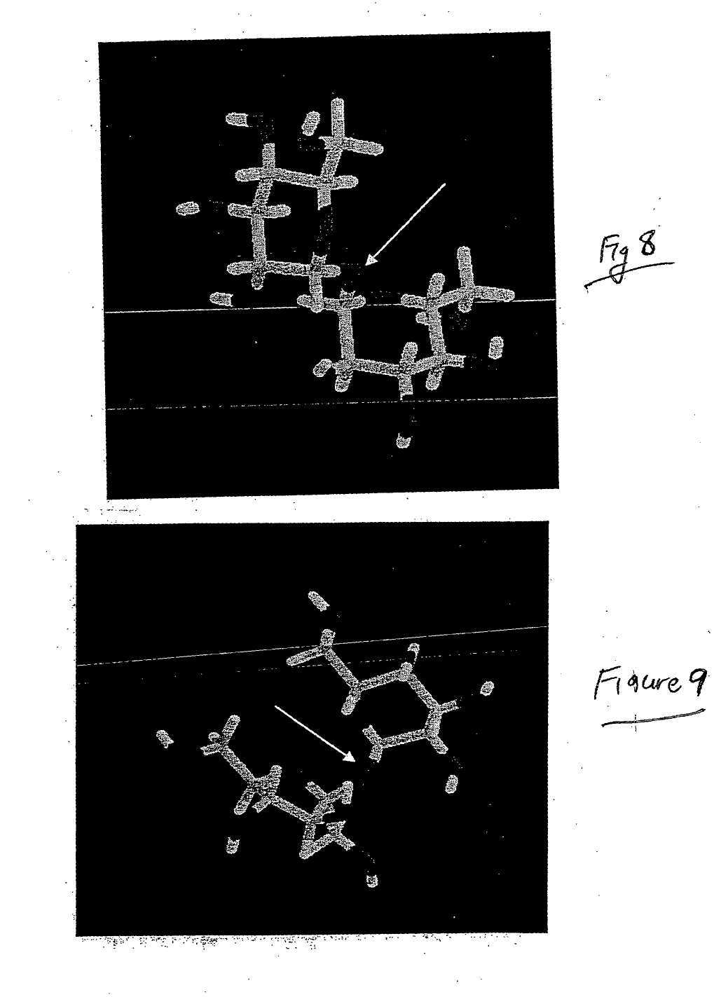

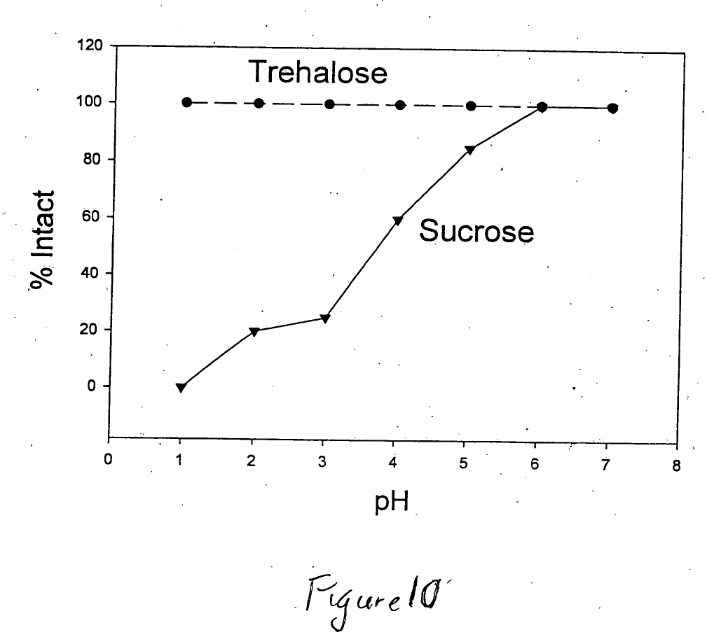

The biological sample 100 is disposed in a solute solution 126 having a solute T (e.g., trehalose). As shown in FIG. 2, the solute T is transferred or passed in direction of the arrow A from the solute solution 126 against and/or into a portion of the membrane 104. As previously indicated, the solute solution 126 may be heated to an elevated temperature (e.g., a temperature from about 30° C. to about 40° C.) to assist in transferring the solute T out of the solute solution 126 and against and/or into a portion of the membrane 104, causing the plasma membrane 104 including its associated protein coat 105 to bulge and/or concave inwardly (as best shown in FIG. 3) to begin the formation of a portion of the membrane 104 having the solute T; that is, a vesicle 120 (see FIG. 4) begins to form. Referring now to FIG. 5 these is seen a partial plan view of the biological sample 100 after the subsequent release or “budding off” of the vesicle 120 into the cytoplasm 108. The vesicle 120 is coated with the protein 105 and contains the solute T. As exemplarily shown in FIG. 6, the vesicle 120 fuses with lysosome 112 to produce and/or form fused matter 124 which is also coated with the protein 105.

The internal, engulfed material within the fused matter 124 contains a reduced pH (e.g., a pH ranging from about 3.5 to about 6.0) due to ion pumps in the membrane. The acid hydrolases are activated by the low pH. The reduced pH of the internal, engulfed material causes the outer skin or membrane of the produced fused matter 124 to have an increased permeability which facilitates the leakage or passage of the solute (or any low molecular weight molecules) through the outer skin or membrane of the fused matter 124, as illustrated in FIG. 7. As previously indicated, when the solute is trehalose or any other low molecular weight molecule that is immune to the acidic engulfed material within the fused matter 124, trehalose escapes degradation due to the stability of its associated glycosidal linkage and freely passes intact through the increased-permeability membrane of the fused matter. As previously suggested, the remaining broken down components of the lysosome and the vesicle are released into the cytoplasm for further metabolism. Thus, the solute T is transferred out of the fused matter 124, as represented by arrow B in FIG. 7, when the permeability of the membrane of the fussed matter 124 is increased, and when the engulfed material within the fused matter 124 breaks down or degrades for further metabolism within the cytoplasm. As previously indicated, the solute T preferably remains intact during the loading and/or solute transferring process and within the internal environment of the fused matter 124. Thus, the solute T remains essentially intact and whole when transferred out of the fused matter 124 and into the cytoplasm 108. The solute T survives conditions found in the lysosome-endosome and the intact solute T leaks through the outer membrane of the fused matter 124 and into the cytoplasm. The biological sample 100 is now ready for further processing, such as drying, freezing, and subsequent rehydration, etc.

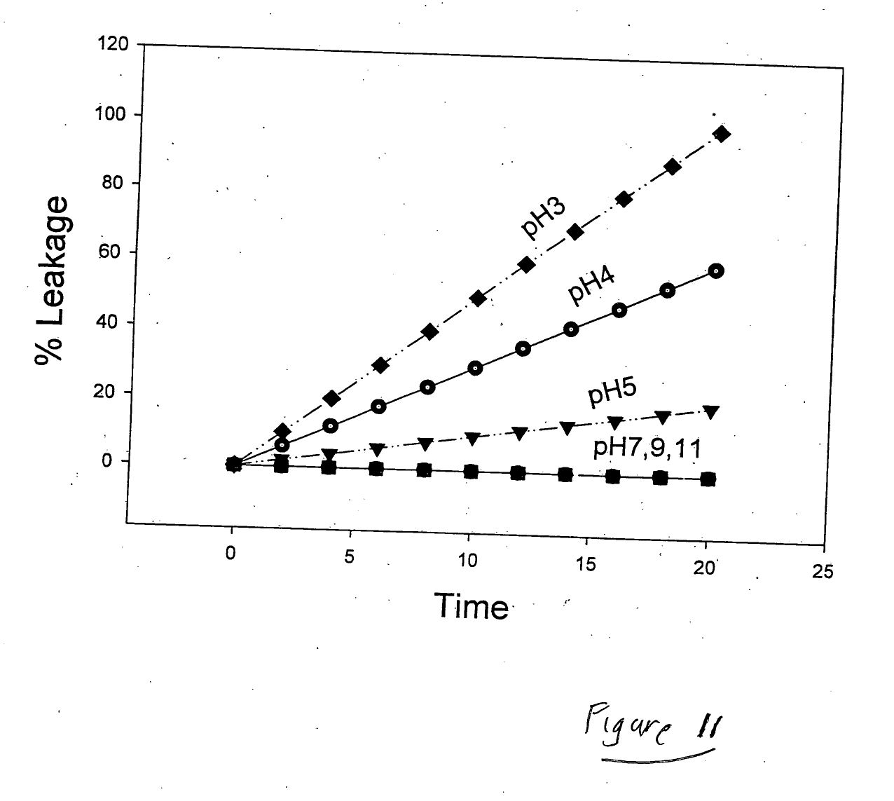

A preferred solute for embodiments of the present invention comprises trehalose. Most sugars degrade in fused lysosome-endosome due to the reduced pH and presence of acid hydrolases. Trehalose is the only non-reducing disaccharide of glusose. FIG. 8 is an enlarged chemical structural, chain formula diagram of trehalose, a non-reducing disaccharide of glucose, with an arrow pointing to a glycosidic bond. Severing of the glycosidic bond produces glucose which is ineffective in stabilizing dry biological materials. Sucrose, on the other hand, is a non-reducing disaccharide of glucose and fructose. FIG. 9 is an enlarged chemical structural, chain formula diagram of sucrose, a non-reducing disaccharide of glucose and fructose, with an arrow pointing to a glycosidic bond which is much more susceptible to hydrolysis than the glycosidic bond in trehalose. Trehalose survives conditions found in the lysosome-endosome and intact trehalose leaks into the cytosol of living cells.

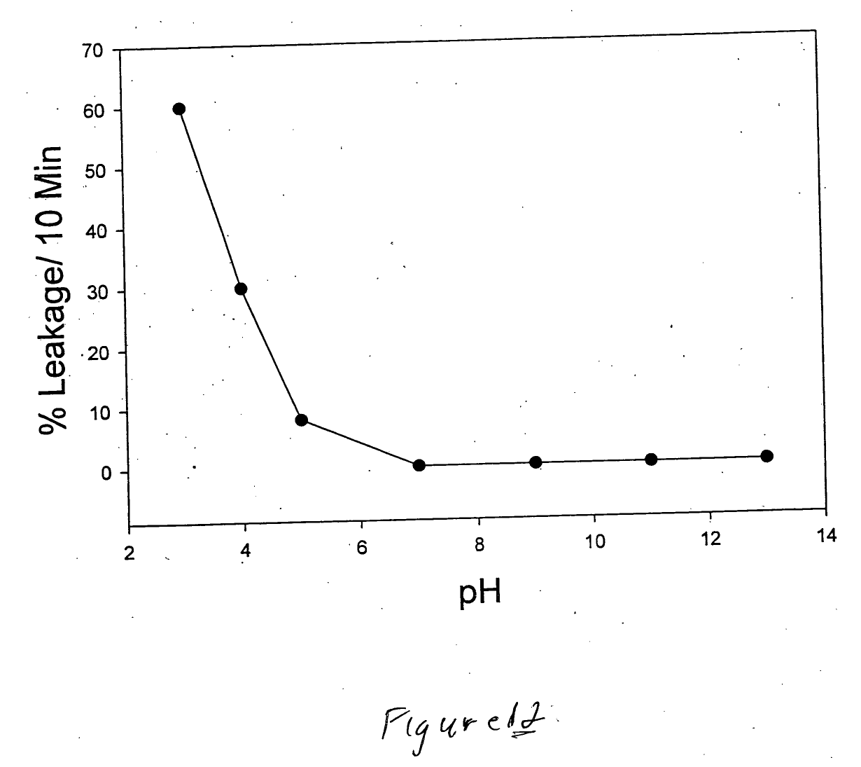

Referring now to FIG. 10, there is seen a graph of pH vs. % intact (i.e., % non-degraded) for trehalose and sucrose. Trehalose survives survival (i.e., remains 100% intact) down to a pH 1, while sucrose hydrolyzes into glucose and fructose at pH as 5. The % of intact sucrose commences to decrease below a pH of about 6. Thus, sucrose begins to break down at a pH below 6. Example 1 below provides the more specific testing conditions and parameters which produced the graphical, illustrations of FIG. 10.

FIG. 11 is a graph of % leakage of a fluorescent dye, carboxyfluorescein (CF), from phospholipid vesicles as a function of pH and time. As the pH decreases from about 7.0 to a pH of about 3.0 and as time increases (e.g., increases from about 0 to about 20 minutes, the % leakage of the fluorescent dye increases. There is little or no leakage at a pH of about 7.0 or above, but leakage proceeds rapidly at a pH below about 5.0. At pH of about 3.0, 100% of the solute leaked out in 20 minutes. Thus, the leakage of the fluorescent dye CF from liposomes increases with pH and time.

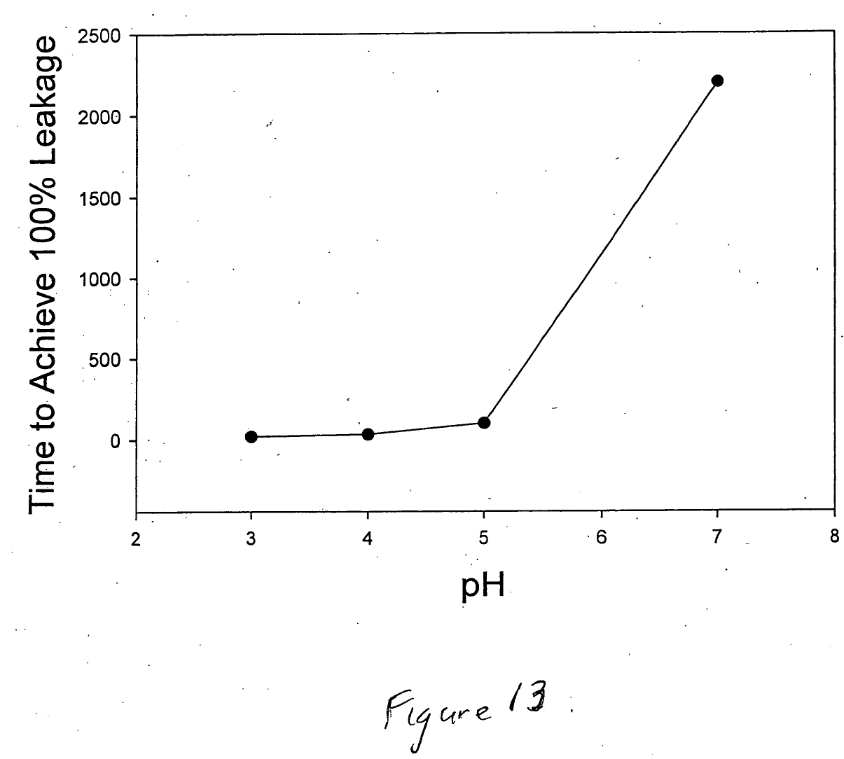

With respect to rate of leakage and the time for leakage, the rate of leakage increases as the pH decreases, as best illustrated in FIG. 12, and the time to achieve 100% leak increases with increase in pH, as best shown in FIG. 13. FIG. 12 is a graph of rates of leakage (% leakage/10 minutes) as a function of pH. At pH of 3-4 leakage goes to completion in 20-30 minutes, while at pH 7, three months would be required to complete the leakage. FIG. 13 is a graph of projected time to achieve 100% leakage, based on FIGS. 11 and 12, as a function of pH. The time to achieve 100% depletion especially increases after a pH of 5. Example 2 below provides the more specific testing conditions and parameters which produced the graphical, illustrations of FIGS. 11-13.

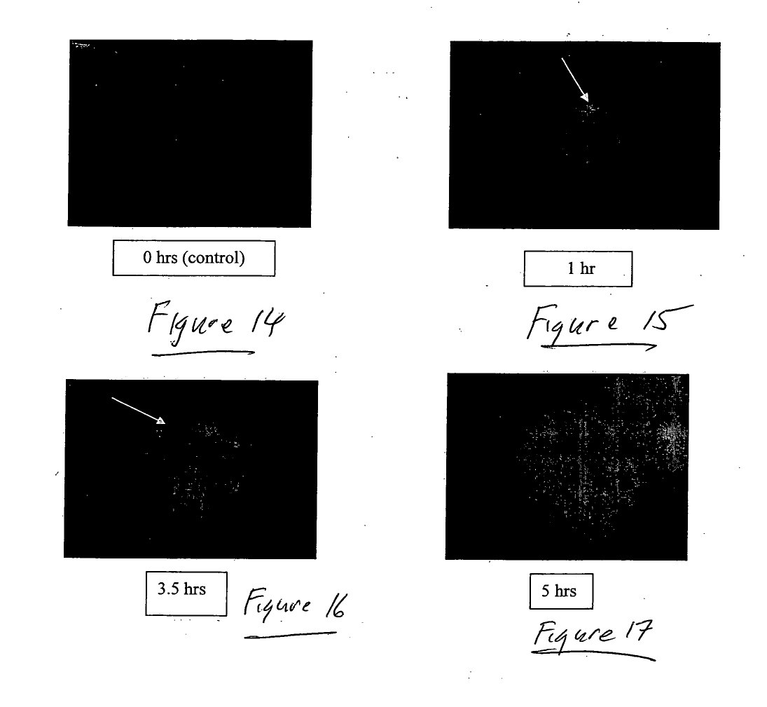

Referring now to FIGS. 14-17, there is seen a distribution of Lucifer yellow in intact cells as a function of incubation time. More specifically, FIG. 14 is a picture of control cells at zero (0) incubation time, showing no leakage of Lucifer yellow dye into the cytoplasm-of the control cell. FIG. 15 is a picture of cells after 1 hour incubation time, showing Lucifer yellow dye in punctate structures (i.e., endocytotic vesicles) with some leakage of Lucifer yellow dye into the cytoplasm. FIG. 16 is a picture of cells after 3.5 hours incubation time, showing Lucifer yellow dye in punctate structures (i.e., endocytotic vesicles) with more leakage of Lucifer yellow dye into the cytoplasm than the leakage represented in the picture of FIG. 15; and FIG. 17 is a picture of cells after 5.0 hours incubation time, showing a uniform stain of Lucifer yellow dye which suggests that Lucifer yellow dye has leaked into the cytoplasm. Example 3 below provides the more specific testing conditions and parameters which produced the graphical, illustrations of FIGS. 14-17. At short incubation times (e.g., incubation times of 1 hour and 3.5 hours), the dye is in punctate structures. With long incubation time (e.g., 5 hours) the staining becomes uniform, suggesting that the dye has leaked into the cytoplasm. Example 3 below provides the more specific testing conditions and parameters which produced the graphical, illustrations of FIGS. 14-17.

Embodiments of the present invention will be illustrated by the following set forth examples which are being given by way of illustration only and not by way of any limitation. It is to be understood that all materials, chemical compositions and procedures referred to below, but not explained, are well documented in published literature and known to those artisans possessing skill in the art.

All materials and chemical compositions whose source(s) are not stated below are readily available from commercial suppliers, who are also known to those artisans possessing skill in the art. All parameters such as concentrations, mixing proportions, temperatures, rates, compounds, etc., submitted in these examples are not to be construed to unduly limit the scope of the invention.

### EXAMPLE 1

Trehalose and sucrose solutions were prepared in water (100 mM). The solutions were heated to 70° C. for 30 minutes, after which the solutions were analyzed by HPLC (high performance liquid chromatography. Trehalose survived this treatement down to pH 1.0, while most of the sucrose was hydrolyzed to glucose and fructose at pH as high as 5. At lower temperatures this pattern persisted, although the time required to hydrolyze the sucrose increased. It is well established that the pH in lysosomes is 4-5, so it follows that sucrose if likely to be degraded in lysosomes, while trehalose should escape damage. The residence time in the lysosomes would be expected to be critical in this regard. At 37° C., for example, sucrose would experience minimal degradation if the residence time is 10 minutes, but degradation would be extensive if the residence time were on the order of hours.

### EXAMPLE 2

Membranes become leaky at the pH found in lysosomes. Liposomes composed of the phospholipids POPC (palmitoyloleyoylphosphatidylcholine) and PS (phosphatidylserine) (9:1) were prepared by extrusion through 100 nm filters. A marker for permeability, the fluorescent marker carboxyfluorescein (CF) was trapped in liposomes at a concentration of 0.5 M during the extrusion. External CF was removed by passing the liposomes through a Sephadex column. The liposomes were then subjected to decreased pH. CF is fluorescent, but self-quenching at the concentration at which it was trapped in the lipsosomes. When the trapped CF leaks into the external medium, it becomes diluted, and fluorescence increases. From the rate of increase in fluorescence it is possible to deduce the permeability.

### EXAMPLE 3

Leakage from lysosomes in vivo is in reasonable agreement with the in vitro data. Cells were incubated in a fluorescent probe, Lucifer yellow. This particular probe was chosen as a tracer since it is approximately the same size as a disaccharide. The cells were washed free of extracellular Lucifer yellow and then observed by fluorescence microscopy. The results are shown in FIGS. 14-17. When the cells were incubated in the dye for 1 to 3.5 hours, punctuate staining was clearly seen, indicating the presence of the dye in endosomes or lysosomes. However, by 5 hours much of the punctuate staining disappeared and the cytoplasm acquired a uniform fluorescence. Thus, 3.5 to 5 hours are required for appreciable leakage to occur. Thus, there is reasonable agreement between the two measurements.

### EXAMPLE 4

Trehalose survives passage through lysosomes in vivo, while other sugars do not. Platelet cells were incubated for four hours in 100 mM trehalose, sucrose, or raffinose, respectively. The platelet cells were then homogenized in 60% methanol, from which the large particles were pelleted by centrifugation. The supernatant was removed, and analyzed by HPLC. The results showed that trehalose was recovered intact, with no evidence of degradation. Raffinose appeared to be completely hydrolyzed. Sucrose was partially hydrolyzed, but significant amounts of intact sucrose were obtained, nevertheless. It may well be that the difference between raffinose and sucrose lies in the fact that raffinose is a trisaccharide and thus might be expected to leak across the lysosomal membrane more slowly than does sucrose. Thus, with increased residence time hydrolysis would go further towards completion. Even a small amount of hydrolysis might not be acceptable; the monosaccharides that are produced as a result of the hydrolysis are all reducing sugars, and all show the Maillard reaction with dry proteins, a reaction that denatures the protein irreversibly.

### Conclusion

Embodiments of the present invention provide that trehalose, a sugar found at high concentrations in organisms that normally survive dehydration, can be used to preserve biological structures in the dry state. Human biological sample(s) can be loaded with trehalose under specified conditions, and the loaded biological sample(s) can be dried (e.g., freeze dried) with excellent recovery.

While the present invention has been described herein with reference to particular embodiments thereof, a latitude of modification, various changes and substitutions are intended in the foregoing disclosure, and it will be appreciated that in some instances some features of the invention will be employed without a corresponding use of other features without departing from the scope and spirit of the invention as set forth. Therefore, many modifications may be made to adapt a particular situation or material to the teachings of the invention without departing from the essential scope and spirit of the present invention. It is intended that the invention not be limited to the particular embodiment disclosed as the best mode contemplated for carrying out this invention, but that the invention will include all embodiments and equivalents falling within the scope of the appended claims.

1. A process for loading a biological sample comprising; loading a biological sample with a solute by fluid phase endocytosis to produce an internally loaded biological sample.

2. The process of claim 1 wherein said loading a biological sample by fluid phase endocytosis comprises fusing within the biological sample a first matter with a second matter to produce a fused matter.

3. The process of claim 2 wherein said first matter comprises the solute.

4. The process of claim 2 wherein said first matter comprises a vesicle having the solute.

5. The process of claim 2 wherein said second matter comprises a lysosome.

6. The process of claim 4 wherein said second matter comprises a lysosome.

7. The process of claim 2 wherein said fused matter comprises the solute.

8. The process of claim 6 wherein said fused matter comprises the solute.

9. The process of claim 2 wherein said loading a biological sample by fluid phase endocytosis additionally comprises transferring the solute from the fused matter within the biological sample.

10. The process of claim 8 wherein said loading a biological sample by fluid phase endocytosis additionally comprises transferring the solute from the fused matter within the biological sample.

11. The process of claim 9 wherein the solute is transferred from the fused matter into a cytoplasm within the biological sample.

12. The process of claim 10 wherein the solute is transferred from the fused matter into a cytoplasm within the biological sample.

13. The process of claim 2 wherein said fused matter comprises a lower pH than a pH of the first matter.

14. The process of claim 12 wherein said fused matter comprises a lower pH than a pH of the first matter.

15. The process of claim 2 wherein said fused matter comprises a pH of less than about 6.5.

16. The process of claim 1 wherein said biological sample includes a biological sample selected from a group of biological samples comprising a platelet and a cell.

17. The process of claim 1 wherein said solute comprises trehalose.

18. A biological sample produced in accordance with the process of claim 1.

19. A process for preparing a dehydrated biological sample comprising:
providing a biological sample selected from a mammalian species;
loading the biological sample with a solute by fluid phase endocytosis to produce a loaded biological sample; and
drying the loaded biological sample to produce a dehydrated biological sample.

20. The process of claim 19 wherein said loading of the biological sample with a solute comprises loading of the biological sample with an oligosaccharide from an oligosaccharide solution.

21. A process for loading a solute into a biological sample comprising:
forming within a biological sample a vesicle having a solute; and
lowering the pH of the vesicle to cause the biological sample to be loaded with the solute.

22. The process of claim 21 wherein said lowering of the pH of the vesicle comprises fusing the vesicle with a lysosome to produce fused matter.

23. The process of claim 21 wherein said lowering of the pH of the vesicle comprises increasing the permeability of a membrane in the biological sample for facilitating the passage of the solute from the vesicle into the biological sample.

24. The process of claim 22 wherein said fused matter comprises a pH of less that about 6.5.

25. A biological sample produced in accordance with the process of claim 21.
